(19) United States
(12) Patent Application Publication
(10) Pub. No.: US 2023/0055044 A1
(43) Pub. Date: Feb. 23, 2023

(54) METHOD FOR MANUFACTURING INDUCED PLURIPOTENT STEM CELLS

(71) Applicants: Koji TANABE, Palo Alto, CA (US); I Peace, Inc., Palo Alto, CA (US)

(72) Inventors: Koji TANABE, Palo Alto, CA (US); Kenta SUTO, Palo Alto, CA (US)

(73) Assignee: I Peace, Inc., Palo Alto, CA (US)

(21) Appl. No.: 17/759,315

(22) PCT Filed: Jan. 22, 2021
(86) PCT No.: PCT/JP2021/002337

§ 371 Date: Jul. 22, 2022

Related U.S. Application Data
(60) Provisional application No. 62/988,131, filed on Mar. 11, 2020.
(60) Provisional application No. 62/965,669, filed on Jan. 24, 2020.
(60) Provisional application No. 63/131,451, filed on Dec. 29, 2020.

(51) Int. Cl. C12N 5/074; C12N 5/10; C12N 15/88

(52) U.S. Cl. CPC C12N 5/0696; C12N 5/10; C12N 15/88; C12N 2501/602; C12N 2501/603; C12N 2501/604; C12N 2501/606; C12N 2533/52; C12N 2502/11; C12N 2502/253

(57) ABSTRACT

According to the present disclosure, provided is a method for manufacturing induced pluripotent stem cells including preparing cells and introducing RNA into the cells, wherein the RNA includes RNA encoding a reprogramming factor and wherein, in the RNA introduced into the cells, double-stranded RNA is substantially removed.

### TECHNICAL FIELD

The present invention relates to a cell technique and a method for manufacturing induced pluripotent stem cells.

### BACKGROUND ART

Induced pluripotent stem (iPS) cells are cells having two characteristic abilities. One is an ability to transform into all the cells that constitute the body. The other is to have a semi-permanent proliferative ability. Since iPS cells have these two abilities, they can be applied to a transplantation treatment without being rejected by producing iPS cells from their own somatic cells and transforming them into target somatic cells. Therefore, iPS cells are considered to be a key technology for regenerative medicine.

From the creation of iPS cells to the present, many methods for manufacturing iPS cells have been established. Examples of typical methods for manufacturing iPS cells include a method using retroviruses/lentiviruses and a method using an episomal vector.

The method using retroviruses/lentiviruses will be described. Somatic cells are infected with retroviruses or lentiviruses, and genes encoding an initialization factor can be introduced into cells. In addition, retroviruses or lentiviruses allow an initialization factor to be inserted into the genome of somatic cells and induce stable expression of the initialization factor in the cells.

However, the method using retroviruses/lentiviruses has the following problems. First, insertion of the initialization factor into the genome of somatic cells may damage existing genes and promoters, which can cause canceration of the cells. In addition, the initialization factor inserted into the genome may be reactivated after iPS cells are transformed to somatic cells. Therefore, cells for transplantation derived from iPS cells have a risk of canceration. Actually, in a mouse model, reactivation of the introduced initialization factor is observed in somatic cells, and canceration has been confirmed (for example, refer to Non-Patent Document 1).

An episomal vector is circular DNA and self-amplified in the nucleus (for example, refer to Patent Document 1). It had been thought that an episomal vector is not integrated into the genome in principle, but in recent studies, it has been reported that fragments of episomal vectors are scattered and inserted into the genome of iPS cells produced using an episomal vector. Therefore, there is a problem of the reprogramming gene remaining in the cells. For example, if C-MYC or KLF4 remains in the cells, it causes canceration. It is expensive to examine whether the reprogramming gene remains in cells. It is not possible to show that all cells in the transplanted cell pool have no insertion of the episomal plasmid into the genome and have no residue.

Since the method using retroviruses/lentiviruses and the method using an episomal vector have the above problems, a method for manufacturing iPS cells using RNA has been proposed (for example, refer to Non-Patent Document 2).

### CITATION LIST

### Patent Document

Patent Document 1: Japanese Patent No. 5376478

### Non-Patent Document

Non-Patent Document 1: Nature 448, 313-317
Non-Patent Document 2: Nature Biotechnol 26(3): 313-315, 2008.

### SUMMARY

### Technical Problem

One object of the present invention is to provide a method for efficiently manufacturing induced pluripotent stem cells without being limited to a method for introducing a reprogramming factor.

### Solution to Problem

According to an aspect of the present invention, there is provided a method for manufacturing induced pluripotent stem cells including preparing cells; and introducing RNA into the cells, wherein the RNA includes RNA encoding a reprogramming factor, and wherein, in the RNA, double-stranded RNA is substantially removed.

In the method, the RNA may be purified through HPLC.

In the method, the RNA may further include RNA encoding a dominant negative mutant of p53.

In the method, the RNA encoding the reprogramming factor may include at least one selected from the group consisting of OCT3/4 RNA, SOX2 RNA, KLF4 RNA, and C-MYC RNA.

In the method, the RNA encoding the reprogramming factor may include OCT3/4 RNA and RNA of a transactivation domain (TAD) of MYOD connected to the OCT3/4 RNA.

In the method, the RNA may be introduced into the cells by a lipofection method.

In the method, in the introduction, the cells may be adhered to a substrate.

In the method, the substrate may be coated with a matrix in which induced pluripotent stem cells are able to be cultured.

In the method, the cells may be fibroblasts.

In the method, the cells may be blood cells.

In the method, the blood cells may not be expansion-cultured endothelial progenitor cell.

In the method, before the RNA is introduced, the blood cells may be expansion-cultured in a blood cell medium for 10 days or less.

In the method, the blood cells may be at least one selected from the group consisting of mononuclear cells, T cells, B cells, monocytes, macrophages, blood stem cells, dendritic cells, and granulocytes.

In the method, the blood cells may be derived from peripheral blood or cord blood.

In the method, the blood cells may be derived from adult blood.

The method may further include culturing the blood cells into which the RNA is introduced in a blood cell medium and then culturing in a stem cell induction medium.

In the method, the cells may be cells contained in urine.

In the method, the cells may be bladder epithelial cells.

The method may further include collecting cells into which the RNA is introduced from urine.

In the method, the cells may be derived from humans.

In the method, the cells may be derived from non-human primate animals.

### Advantageous Effects of Invention

According to the present invention, it is possible to provide a method for efficiently manufacturing induced pluripotent stem cells.

### BRIEF DESCRIPTION OF DRAWINGS

FIG. 1 is a diagram showing the results obtained by dot-blot analyzing M30 RNA using anti-double-stranded RNA antibody J2 in Example 1.

FIG. 2 is a diagram showing the results obtained by dot-blot analyzing SOX2 RNA using anti-double-stranded RNA antibody J2 in Example 1.

FIG. 3 is a diagram showing the results obtained by dot-blot analyzing C-MYC RNA using anti-double-stranded RNA antibody J2 in Example 1.

FIG. 4 is a diagram showing the results obtained by dot-blot analyzing KLF4 RNA using anti-double-stranded RNA antibody J2 in Example 1.

FIG. 5 is a diagram showing the results obtained by dot-blot analyzing LIN28 RNA using anti-double-stranded RNA antibody J2 in Example 1.

FIG. 6 shows microscope images of the results of Example 1.

FIG. 7 is a dot plot showing the results of Example 1 obtained by a flow cytometer.

FIG. 8 is an image showing gene expression analysis results according to Example 1.

FIG. 9 is an image showing gene expression analysis results according to Example 1.

FIG. 10 is a dot plot showing the results of Comparative Example 1 obtained by a flow cytometer.

FIG. 11 is a diagram showing the results obtained by dot-blot analyzing p53P275S RNA using anti-double-stranded RNA antibody J2 in Example 2.

FIG. 12 is a graph showing the number of iPS-cell-like colonies according to Example 2.

FIG. 13 shows microscope images of the results of Example 3.

FIG. 14 is a dot plot showing the results of Example 3 obtained by a flow cytometer.

FIG. 15 is a microscope image showing the results of Comparative Example 2.

FIG. 16 is a graph showing the results of Example 4.

FIG. 17 shows graphs showing gene expression according to Example 5.

FIG. 18 is a microscope image showing the results of Example 6.

FIG. 19 shows microscope images of the results of Example 6.

FIG. 20 is a dot plot showing the results of Example 6 obtained by a flow cytometer.

FIG. 21 is a microscope image showing the results of Example 7.

FIG. 22 shows microscope images of the results of Example 7.

FIG. 23 is a dot plot showing the results of Example 7 obtained by a flow cytometer.

FIG. 24 is an image of urine-derived cells according to Example 8.

FIG. 25 is an image of urine-derived cells according to Example 8.

FIG. 26 shows images of urine-derived cells transfected with RNA encoding GFP according to Example 9.

FIG. 27 shows images of urine-derived cells into which the reprogramming factor is introduced according to Example 10.

FIG. 28 is an image of urine-derived cells into which the reprogramming factor is introduced according to Example 11.

FIG. 29 shows dot plots obtained by a flow cytometer according to Example 12.

FIG. 30 shows images of urine-derived cells into which the reprogramming factor is introduced according to Example 13.

FIG. 31 shows images of urine-derived cells into which the reprogramming factor is introduced according to Example 13.

### DESCRIPTION OF EMBODIMENTS

Embodiments of the present invention will be described below in detail. Here, the following embodiments exemplify a device and a method for embodying the technical ideas of the invention, and the technical ideas of the invention do not specify the combination of constituent members or the like as in the following. The technical ideas of the invention can be variously modified within the scope of the claims.

A method for manufacturing induced pluripotent stem cells (iPS cells) according to an embodiment includes preparing cells and introducing RNA into the cells. The RNA introduced into cells may include RNA encoding a reprogramming factor and RNA encoding a dominant negative mutant of p53.

RNA introduced into cells is, for example, single-stranded RNA, from which double-stranded RNA is substantially removed. In addition, RNA introduced into cells is preferably substantially free of impurities such as small RNA and contaminants. The single-stranded RNA introduced into cells may be purified and/or concentrated in order to substantially remove double-stranded RNA. Examples of a method for purifying single-stranded RNA introduced into cells include a purification method using high performance liquid chromatography (HPLC). For example, 70% or more, 75% or more, 80% or more, 85% or more, or 90% or more of double-stranded RNA is removed through HPLC. Alternatively, in order to substantially remove double-stranded RNA, RNA introduced into cells may be treated with ribonuclease that decomposes double-stranded RNA. RNA introduced into cells is, for example, mRNA.

p53 is a cancer-suppression protein. The dominant negative mutant of p53 is not particularly limited as long as it can act competitively with a wild type p53 protein in somatic cells and inhibit a function of the wild type p53 protein. Examples of dominant negative mutants of p53 include p53P275S in which proline at position 275 (at position 278 in the case of humans) located in a DNA binding region of mouse p53 is point-mutated to serine, p53DD in which an amino acid at position 14-301 of mouse p53 (corresponding to position 11-304 in human p53) is deficient, p53S58A in which serine at position 58 of mouse p53 (at position 61 in the case of humans) is point-mutated to alanine, p53C135Y in which cysteine at position 135 of human p53 (at position 132 in the case of mice) is point-mutated to tyrosine, p53A135V in which alanine at position 135 of mouse p53 (at position 138 in the case of humans) is point-mutated to valine, p53R172H in which arginine at position 172 of mouse p53 (at position 175 in the case of humans) is point-mutated to histidine, p53R270H in which arginine at position 270 of mouse p53 (at position 273 in the case of humans) is point-mutated to histidine, and p53D278N in which aspartic acid at position 278 of mouse p53 (at position 281 in the case of humans) is point-mutated to asparagine.

RNA introduced into cells may further include RNA in the transactivation domain (TAD) of MYOD that is directly connected to the full length of OCT3/4 RNA.

The RNA may be introduced into the cells by a lipofection method. When RNA is used, it is possible to reduce a dead cell rate in the procedure of lipofection and improve induction efficiency. Alternatively, the RNA may be added to a medium and the RNA may be naturally incorporated into the cells.

Here, inducing cells to iPS cells may be referred to as cell reprogramming or cell initialization. In the present disclosure, although the gene symbols are denoted as those of humans, this is not intended to limit the species by uppercase or lowercase letters. For example, denoting in all uppercase letters does not exclude inclusion of mouse or rat genes. However, in the examples, the gene symbols are shown according to the species actually used.

Examples of cells into which RNA is introduced include somatic cells such as blood cells, fibroblasts, dental pulp stem cells, keratinocytes, hair papilla cells, oral epithelial cells, and somatic stem progenitor cells. The cells into which RNA is introduced may be cells contained in urine. Examples of cells contained in urine include bladder epithelial cells. The cells may be derived from humans or may be derived from non-human animals. The non-human animals may be non-human primates. Examples of non-human primates include chimpanzees, bonobos, gorillas, orangutans, gibbons, guenons, platyrrhines, and prosimians.

Blood cells are isolated from blood. The blood is, for example, peripheral blood or cord blood, but is not limited thereto. Blood may be collected from an adult or a minor. During blood sampling, an anticoagulant such as ethylene-diamine-tetraacetic acid (EDTA), heparin, and a biological preparation standard blood preservative solution A (ACD-A) is used.

Blood cells include, for example, nucleated cells such as mononuclear cells including monocytes and lymphocytes, macrophages, blood stem cells, dendritic cells, granulocytes, neutrophilic leukocytes, eosinophilic leukocytes, and basophil leukocytes. Blood cells are, for example, T cells and B cells. T cells are, for example, αβT cells. Here, the blood cells may not include expansion-cultured endothelial progenitor cells (EPC), red blood cells, and platelets. Alternatively, before RNA is introduced, blood cells may be expansion-cultured in a blood cell medium for 10 days or less, 7 days or less, 5 days or less, 4 days or less, 3 days or less, 2 days or less, or 1 day or less.

Mononuclear cells are isolated from blood using a medium for isolating blood cells, a centrifugal device or the like. A method for isolating mononuclear cells when Ficoll (GE Healthcare) is used as a medium for isolating blood cells is as follows.

Since the isolation accuracy of mononuclear cells tends to deteriorate at a low temperature, the centrifuge is set at 4° C. to 42° C., preferably 18° C. 10 μL to 50 mL of blood is sampled from an adult or minor human, a chelating agent containing EDTA is added to the blood to prevent the blood from coagulating, and is mixed gently. In addition, 5 mL of a medium for isolating human lymphocytes (Ficoll-Paque PREMIUM, GE Healthcare Japan) is dispensed into two 15 mL tubes. 5 mL of PBS is added to 5 mL of blood for dilution, and 5 mL layers are placed on the medium for isolating human lymphocytes in each of the tubes. In this case, the diluted blood is slowly added onto the medium along the tube wall of the tube to prevent disturbance of the interface.

The solution in the tube is centrifuged at 10×g to 1,000×g, and preferably, 400×g, at 4° C. to 42° C., preferably 18° C., for 5 minutes to 2 hours, preferably for 30 minutes. After centrifugation, a cloudy white intermediate layer appears in the tube. The cloudy white intermediate layer contains mononuclear cells. The cloudy white intermediate layer in the tube is slowly recovered with a Pipeteman and is transferred to a new 15 mL tube. In this case, the lower layer should not be sucked up. About 1 mL of the cloudy white intermediate layer can be recovered from one tube. Two intermediate layers are transferred together into one tube.

1 mL to 48 mL, preferably 12 mL of PBS is added to the recovered mononuclear cells, and the solution is additionally centrifuged at 10×g to 1,000×g, preferably 200×g, at 4° C. to 42° C., preferably 18° C., for 1 minute to 60 minutes, preferably 10 minutes. Then, the supernatant of the solution is sucked up and removed using an aspirator, and 1 mL to 12 mL, preferably 3 mL of a serum-free hematopoietic cell medium of known composition (X-VIVO (registered trademark) 10, Lonza) is added for suspension therein to obtain a mononuclear cell suspension. Of this, 10 μL of a mononuclear cell suspension is stained with trypan blue and counting is performed on a hemacytometer.

A method for isolating mononuclear cells when a Vacutainer (registered trademark, BD) is used as a blood collection tube is as follows.

Since the isolation accuracy of mononuclear cells tends to deteriorate at a low temperature, the centrifuge is set to 4° C. to 42° C., preferably 18° C. 8 mL of blood is sampled from an adult or minor human using a blood collection tube (Vacutainer (registered trademark), BD), mixed by inversion and mixed with an anticoagulant. Then, the balance is adjusted, and the solution is centrifuged at 4° C. to 42° C., preferably 18° C., at 100×g to 3,000×g, preferably 1,500×g to 1,800×g with a swing rotor for 1 minute to 60 minutes, preferably 20 minutes. After centrifugation, the upper layer, which is a plasma layer, is removed and pipetting is performed to suspend the mononuclear cell layer and blood cells adhered to the gel to obtain a suspension. The obtained suspension is transferred to another 15 mL tube.

1 mL to 14 mL, preferably 12 mL of PBS is added to the suspension in a 15 mL tube, and the suspension is centrifuged at 4° C. to 42° C., preferably 18° C., at 100×g to 3,000×g, preferably 200×g for 1 minute to 60 minutes, preferably 5 minutes. After centrifugation, the supernatant is removed with an aspirator. In addition, a hemolytic agent (PharmLyse (registered trademark), 10-fold concentration, BD) is diluted to a 1-fold concentration with sterilized water. The pellet in the 15 mL tube is loosened by tapping, and 1 mL to 14 mL, preferably 1 mL of a hemolytic agent is added. Then, light is blocked therefrom and the solution is left for 1 minute to 60 minutes, preferably 1 minute at room temperature.

Next, 1 mL to 14 mL, preferably 12 mL of PBS is added to a 15 mL tube, and centrifugation is performed at 4° C. to 42° C., preferably room temperature, at 100×g to 3,000×g, preferably 200×g for 1 minute to 60 minutes, or 5 minutes. After centrifugation, the supernatant is removed with an aspirator, and 1 mL to 15 mL, and preferably 3 mL of a serum-free hematopoietic cell medium of known composition (X-VIVO (registered trademark) 10, Lonza) is added for suspension therein to obtain a mononuclear cell suspension. Of this, 10 μL of a mononuclear cell suspension is stained with trypan blue and counting is performed on a hemacytometer.

In addition, CTL-UP1 (commercially available from Cellular Technology Limited), PBMC-001 (commercially available from Sanguine Biosciences), or the like may be used as mononuclear cells.

Alternatively, regarding the blood cells, blood cells that are cryopreserved using a cell cryopreservation solution such as Cellbanker 1, Stem-Cellbanker GMP grade, or Stem-Cellbanker DMSO free GMP grade (ZENOAQ) may be thawed and used.

When thawing mononuclear cells, first, 1 mL to 15 mL, preferably 8 mL of a serum-free hematopoietic cell medium of known composition (X-VIVO (registered trademark) 10, Lonza) is put into a 15 mL tube, the tube containing frozen mononuclear cells is placed in a warm bath at 4° C. to 42° C., preferably 37° C., and the mononuclear cells start to melt. Then, with the remaining ice, the tube containing mononuclear cells is pulled out of the warm bath, and the mononuclear cells are transferred to a tube containing a serum-free hematopoietic cell medium of known composition. Of this, 10 μL of a mononuclear cell suspension is stained with trypan blue and counting is performed on a hemacytometer.

Blood cells may be isolated based on a cell surface marker. T cells are positive for any of CD3, CD4, and CD8. B cells are positive for any of CD10, CD19, and CD20. Macrophages are positive for any of CD11b, CD68, and CD163. Monocytes are positive for any of CD14, CD16, and CD64. T cells and B cells are isolated from blood cells using, for example, an automatic magnetic cell isolating device and immunomagnetic beads. Alternatively, mononuclear cells isolated in advance may be prepared. However, blood cells that are not isolated based on a cell surface marker may be used.

The cells into which RNA is introduced can be adherently cultured on a substrate coated with a matrix in which iPS cells can be cultured such as a basement membrane matrix, for example, Matrigel (Corning), CELLstart (registered trademark, ThermoFisher SCIENTIFIC), vitronectin, or Laminin 511 (iMatrix-511MG or iMatrix-511 silk, nippi) in a feeder-free manner. Laminin can be used as a preferable matrix.

The somatic cells into which RNA is introduced are first cultured in a medium suitable for the type of somatic cells or differentiated cells, which is not a stem cell medium. For example, when the cells are blood cells, the cells are adherently cultured in a serum-free medium for blood cells such as StemSpan (registered trademark) H3000 (STEMCELL TECHNOLOGIES), Human Blood Cell Culture Medium Kit (Cell Applications), X-VIVO (registered trademark) 10 (Lonza), StemSpan (registered trademark) ACF (STEMCELL TECHNOLOGIES), X-VIVO (registered trademark) 15 Serum-free Hematopoietic Cell Medium (Lonza), X-Vivo10 Serum-free Hematopoietic Cell Medium (Lonza), Hematopoietic Progenitor Expansion Medium DXF (Promocell), Hematopoietic Progenitor Medium (Promocell), MethoCult H4434 Classic (registered trademark, STEMCELL TECHNOLOGIES), StemPro-34 SFM (1×) (registered trademark, Thermo Fisher SCIENTIFIC), StemMACS HSC Expansion Media, human (registered trademark, Miltenyi Biotec), StemSpan (registered trademark) SFEM (STEMCELL TECHNOLOGIES), StemSpan (registered trademark) SFEM II (STEMCELL TECHNOLOGIES), CellXVivo Human M1 Macrophage Differentiation Kit (R&D SYSTEMS), and Stemline (registered trademark) II Hematopoietic Stem Cell Expansion Medium (SIGMA-AlDRICH). However, the somatic cells into which RNA is introduced may be suspension-cultured.

The RNA introduced into cells may be modified with at least one selected from the group consisting of pseudouridine (W), 5-methyluridine (5meU), N1-methylpseudouridine (melW), 5-methoxyuridine (5moU), 5-hydroxymethyluridine (5hmU), 5-formyluridine (5fU), 5-carboxymethyl ester uridine (5camU), thioguanosine (thG), N4-methylcytidine (me4C), 5-methylcytidine (m5C), 5-methoxycytidine (5moC), 5-hydroxymethylcytidine (5hmC), 5-hydroxycytidine (5hoC), 5-formylcytidine (5fC), 5-carboxylcytidine (5caC), N6-methyl-2-aminoadenosine (m6DAP), diaminopurine (DAP), 5-methyluridine (m5U), 2′-O-methyluridine (Um or m2′-OU), 2-thiouridine (s2U), and N6-methyl adenosine (m6A). For example, one or more or all of uridine nucleosides in RNA may be replaced with pseudouridine. RNA in which uridine nucleoside is replaced with pseudouridine tends to be translated at a higher level and for a longer time than RNA in which a uridine nucleoside is not replaced with pseudouridine.

RNA introduced into cells may be purified by treatment with RNaseIII. RNaseIII is derived from, for example, E. coli. RNaseIII digests, for example, double-stranded RNA larger than about 12 base pairs.

The RNA introduced into the cells may be polyadenylated. RNA may be prepared by polyadenylation of (IVT) RNA transcribed in vitro. Polyadenylation includes bringing IVT RNA into contact with, for example, poly(A) polymerase (for example, yeast RNA polymerase or E. coli poly(A) polymerase). Alternatively, RNA may be polyadenylated during in vitro transcription using a DNA template encoding a poly(A) end. Regardless of whether RNA is polyadenylated with a poly(A) polymerase or during IVT of a DNA template, RNA may include a poly A end (for example, a poly A end having 50 to 200 nucleotides, for example, preferably 100 to 200, 150 to 200 nucleotides, or more than 150 nucleotides).

RNA introduced into cells may be capped. In order to improve efficiency of expression in cells, for example, 80% or more of RNA molecules contain caps. For example, RNA introduced into cells is synthesized in vitro by incubating uncapped primary RNA in the presence of a capping enzyme. In addition, for example, the primary RNA used in a capping enzyme reaction is synthesized by in vitro transcription (IVT) of DNA molecules encoding RNA to be synthesized. DNA encoding RNA to be synthesized contains an RNA polymerase promoter to which the RNA polymerase binds and transcription starts therefrom.

RNA introduced into cells may have a 5′cap[m7G(5′)ppp(5′)G] structure. The sequence is a sequence that stabilizes RNA and promotes transcription. 5′triphosphate may be removed from RNA having 5′triphosphate according to a dephosphorylation treatment. RNA introduced into cells may have [3′O-Me-m7G(5′)ppp(5′)G] as Anti-Reverse Cap Analog(ARCA). ARCA is a sequence inserted before a transcription start point, which improves transcription efficiency of RNA.

RNA is introduced into cells, for example, by a lipofection method. The lipofection method is a method in which a complex of a nucleic acid, which is a negatively charged substance, and a positively charged lipid, is formed by an electrical interaction, and the complex is incorporated into cells by endocytosis or membrane fusion. The lipofection method has advantages such as less damage to cells, excellent introduction efficiency, ease of operation, and less time-consumption.

For example, RNA is introduced into cells cultured using an RNA transfection reagent. For example, when cells are mononuclear cells, immediately after mononuclear cells are isolated from blood, RNA may be introduced into the mononuclear cells.

Lipofectamine MessengerMAX (registered trademark, Thermo Fisher SCIENTIFIC) can be used as the RNA transfection reagent. Alternatively, regarding the RNA transfection reagent, for example, a lipofection reagent such as Lipofectamine (registered trademark) RNAiMAX (Thermo Fisher SCIENTIFIC), Lipofectamine StemTransfection Reagent (Thermo Fisher SCIENTIFIC), TransIT (Mirus), mRNA-In (MTI-GlobalStem), Stemfect RNA Transfection Kit (ReproCELL), Jet Messenger (Polyplus), Lipofectamin (registered trademark) 2000, Lipofectamin (registered trademark) 3000, NeonTransfection System (Thermo Fisher SCIENTIFIC), Stemfect RNA transfection reagent (Stemfect), NextFect (registered trademark) RNA Transfection Reagent (BiooSientific), Amaxa (registered product) Human T cell Nucleofector (registered product) kit (Lonza, VAPA-1002), Amaxa (registered product) Human CD34 cell Nucleofector (registered product) kit (Lonza, VAPA-1003), and ReproRNA (registered trademark) transfection reagent (STEMCELL Technologies) may be used.

The number of cells during RNA lipofection is, for example, 1 or more, 1×10¹ or more, 1×10² or more, 1×10³ or more, 0.5×10⁴ or more, 1×10⁴ or more, or 1×10⁵ or more. In addition, the number of cells during RNA lipofection is, for example, 5×10⁸ or less, 1×10⁷ or less, 1×10⁶ or less, 2×10⁵ or less, or 0.5×10⁴ or less.

The cells are seeded in a region having an area of, for example, 1.0 mm² or more, 1.0×10² mm² or more, or 3.8×10² mm² or more. In addition, the cells are seeded in a region having an area of, for example, 20×10³ mm² or less, 5, 5×10³ mm² or less, or 2.1×10³ mm² or less.

Cells into which RNA is introduced are preferably seeded in a medium or an incubator at a low concentration. Here, the low concentration is, for example, 1 cell/cm² or more, 0.05×10⁵ cells/cm² or less, 0.10×10⁵ cells/cm² or less, 0.20×10⁵ cells/cm² or less, 0.30×10⁵ cells/cm² or less, 0.40×10⁵ cells/cm² or less, 0.50×10⁵ cells/cm² or less, 0.60×10⁵ cells/cm² or less, 0.70×10⁵ cells/cm² or less, 0.80×10⁵ cells/cm² or less, 0.90×10⁵ cells/cm² or less, 0.10×10⁶ cells/cm² or less, 0.15×10⁶ cells/cm² or less, 0.20×10⁶ cells/cm² or less, or 0.25×10⁶ cells/cm² or less.

Alternatively, the low concentration is a concentration at which 10 or less cells, 9 or less cells, 8 or less cells, 7 or less cells, 6 or less cells, 5 or less cells, 4 or less cells, 3 or less cells, or 2 or less cells can come into contact with each other and 11 or more cells do not come into contact with each other. Here, there may be a plurality of cell masses in which 10 or less cells come into contact with each other. Alternatively, the state in which the entire bottom surface of the cell container is covered with cells is regarded as 100% confluency, and the low concentration is 6% or less confluency, 5% or less confluency, 4% or less confluency, 3% or less confluency, 2% or less confluency, 1% or less confluency, 0.5% or less confluency, 0.1% or less confluency, 0.05% or less confluency, or 0.01% or less confluency. In addition, alternatively, the low concentration is, for example, a concentration at which single cells do not come into contact with each other in the seeded cells. For example, single cells may be seeded in wells of a well plate. The well plate may be a 12-well plate or a 96-well plate.

A total amount of RNA during lipofection per 1 mL of a culture solution is, for example, 5 ng or more, 50 ng or more, 100 ng or more, 200 ng or more, 400 ng or more, or 1 μg or more at one time. In addition, a total amount of RNA during lipofection per 1 mL of a culture solution is, for example, 70 μg or less, 50 μg or less, 10 μg or less, 5 μg or less, 3 μg or less, or 1 μg or less at one time.

The amount of an RNA transfection reagent during lipofection is, for example, 0.1 μL or more, 1.0 μL or more, or 5 μL or more at one time. In addition, the amount of an RNA transfection reagent during lipofection is, for example, 500 μL or less, 100 μL or less, or 40 μL or less at one time.

The time for which RNA lipofection is performed is, for example, 0.1 hours or more, 2 hours or more, 3 hours or more, or 4 hours or more at one time. In addition, the time for which RNA lipofection is performed is, for example, 72 hours or less, 24 hours or less, 12 hours or less, or 6 hours or less at one time.

Introduction of RNA into cells may be performed a plurality of times. Introduction of RNA into cells may be performed, for example, once or one or more times a day or once every two days. Introduction of RNA into cells may be performed, for example, a total of 3 times or more, 5 times or more, 10 times or more, 20 times or more, 25 times or more, 30 times or more, or 35 times or more. In addition, introduction of RNA into cells may be performed, for example, a total of 55 times or less, 50 times or less, 45 times or less, or 40 times or less.

After RNA is first introduced into somatic cells, the cells are cultured in a medium suitable for the type of somatic cells, for example, for 1 day or more, 2 days or more, 5 days or more, or 10 days or more. In addition, after RNA is first introduced into somatic cells, the cells are cultured in a medium suitable for the type of somatic cells, for example, for 50 days or less, 20 days or less, 10 days or less, 7 days or less, or 3 days or less. When the cells are blood cells, after RNA is first introduced into the blood cells, the blood cells are cultured in a blood cell medium, for example, for 1 day or more, 2 days or more, 5 days or more, or 10 days or more. In addition, when the cells are blood cells, after RNA is first introduced into the blood cells, the blood cells are cultured in a blood cell medium, for example, for 50 days or less, 20 days or less, 10 days or less, 7 days or less, or 3 days or less.

After RNA is first introduced into somatic cells, the medium suitable for the type of somatic cells is replaced with a stem cell induction medium after, for example, 1 day or more, 2 days or more, 5 days or more, 10 days or more, or 20 days or more. The stem cell induction medium contains, for example, 10 volume % or more and 30 volume % or less of a serum-free supplement, 0.01 volume % or more and 10 volume % or less of albumin, 0.01 volume % or more and 10 volume % or less of insulin, and 0.01 volume % or more and 10 volume % or less of transferrin. As the serum-free supplement, for example, KnockOut Serum Replacement (registered trademark, ThermoFisher SCIENTIFIC) can be used. When the medium suitable for the type of somatic cells is replaced with a stem cell induction medium, the cells that are adhered to the substrate may remain, and the cells may not be separated from the substrate. After the medium suitable for the type of somatic cells is replaced with a stem cell induction medium, RNA lipofection is continued by the method.

The stem cell induction medium may not contain Wnt. The stem cell induction medium may not contain vascular endothelial growth factor (VEGF). The stem cell induction medium may not contain insulin-like growth factor (IGF). The stem cell induction medium may contain an agent that alleviates a congenital antiviral response of cells such as B18R. The stem cell induction medium may contain albumin. The stem cell induction medium may not contain growth factors FGF such as bFGF and TGF such as TGF-β. Alternatively, the stem cell induction medium may contain FGF such as bFGF, for example, at a low concentration of 4,400 ng/mL or less, 100 ng/mL or less, 50 ng/mL or less, 10 ng/mL or 1 ng/mL or less. In addition, the stem cell induction medium may contain TGF such as TGF-β, for example, at a low concentration of 100 ng/mL or less, 10 ng/mL or less, 5 ng/mL, or 0.5 ng/mL or less. Antibiotics such as penicillin and streptomycin cannot be contained. Here, on the 5th day or 7th day after culture of cells in a stem cell induction medium starts, growth factors may be contained in the stem cell induction medium. The stem cell induction medium may contain human serum or human plasma serum. When the stem cell induction medium is prepared in this manner, it is possible to improve induction efficiency.

The stem cell induction medium may include, for example, at least one selected from the group consisting of ACTH (Sigma), Kenpaullone, leukemia inhibitory factor (LIF), protein kinase C (PKC) inhibitor, PKC inhibitor Go6983, glycogen synthase kinase 3 (GSK-3) inhibitor, GSK-3 inhibitor CHIR99021, fibroblast growth factor (FGF) inhibitor, MAPK inhibitor PD0325901, TGF-β inhibitor, TGF-β receptor inhibitor SB-431542, ROCK inhibitor, Akt activator, vitronectin, N2 supplement (Thermo Fisher SCIENTIFIC), B27 supplement (Thermo Fisher SCIENTIFIC), DMSO, FBS (Thermo Fisher SCIENTIFIC), bovine pituitary extract (BPE), and ascorbic acid.

The stem cell induction medium includes, for example, at least one selected from the group consisting of AlbuMax (Thermo Fisher SCIENTIFIC), bovine serum albumin, human albumin, insulin, polyvinyl alcohol (PVA), platelet-derived growth factor (PDGF), lithium chloride (LiCl), β-mercaptoethanol, p38 inhibitor, phenol red, and Chemically Defined Lipid Concentrate (Thermo Fisher SCIENTIFIC).

The cells are cultured in a stem cell induction medium, for example, for 1 day or more, 2 days or more, 5 days or more, 10 days or more, or 20 days or more, and during this time, RNA is introduced by the method. In addition, the cells are cultured in a stem cell induction medium until formation of iPS-cell-like colonies is observed, and during this time, RNA is introduced by the method. The culture may be adherent culture or suspension-culture. The period until formation of iPS-cell-like colonies is observed is, for example, 3 days or less, 5 days or less, 7 days or less, 10 days or less, 20 days or less, or 40 days or less after the medium suitable for the type of somatic cells is replaced with a stem cell induction medium.

When formation of iPS-cell-like colonies is observed in the stem cell induction medium, RNA lipofection ends, and iPS-cell-like colonies are collected. The collected cells are cultured in a stem cell maintenance medium on a substrate coated with the matrix. The culture may be adherent culture or suspension-culture. Examples of stem cell maintenance media include mTeSR (registered trademark, STEMCELL TECHNOLOGIES) and Stem Fit (registered trademark, Ajinomoto Healthy Supply).

Determination of whether somatic cells have been reprogrammed can be made based on, for example, the morphology of the cells. Alternatively, determination of whether somatic cells have been reprogrammed may be performed by analyzing whether at least one surface marker selected from among cell surface markers TRA-1-60, TRA-1-81, SSEA-1, and SSEA5 which indicate undifferentiation, with a flow cytometer, is positive. TRA-1-60 is an antigen specific for iPS/ES cells, and is not detected in differentiated somatic cells. Since iPS cells can be produced only from TRA-1-60 positive fractions, TRA-1-60 positive cells are considered to be species of iPS cells.

According to the present embodiment, it is possible to produce iPS cells that are integration-free or have no residual exotic genes without damaging the genome of cells. In addition, in the related art, when cells to be initialized are blood cells, it is necessary to expand and culture endothelial progenitor cells and introduce RNA to the expansion-cultured endothelial progenitor cells. On the other hand, according to the present embodiment, since it is not necessary to expand and culture endothelial progenitor cells, for example, RNA can be immediately introduced into isolated mononuclear cells and iPS cells can be induced so that it is possible to shorten the time for producing iPS cells.

### Example 1

Recombinant protein FLT3 ligand (PEPROTECH), recombinant human TPO (PEPROTECH), recombinant human IL-6 (PEPROTECH), recombinant human G-CSF (PEPROTECH), and recombinant human SCF (PEPROTECH) were added to a serum-free medium for blood cells.

The centrifuge was set at 18° C. 5 mL to 50 mL of human adult peripheral blood or human cord blood was collected, and EDTA was added to the blood and mixed gently. In addition, 5 mL of a medium for isolating human lymphocytes (Ficoll-Paque PREMIUM, GE HEALTHCARE JAPAN) was dispensed into two 15 mL tubes. 5 mL of PBS was added to blood for dilution, and 5 mL layers were placed on the medium for isolating human lymphocytes in the tube. In this case, the diluted blood was slowly added onto the medium along the tube wall of the tube to prevent disturbance of the interface.

The solution in the tube was centrifuged at 400×g, 18° C. for 30 minutes. In this case, both acceleration and deceleration were performed slowly. After centrifugation, an intermediate layer containing mononuclear cells appeared in the tube. The intermediate layer in the tube was slowly recovered with a Pipeteman and was transferred to a new 15 mL tube. In this case, the lower layer should not be sucked up. About 1 mL of the intermediate layer could be recovered from one tube. Two intermediate layers were transferred together into one tube.

12 mL of PBS was added to the recovered mononuclear cells, and the solution was additionally centrifuged at 200×g, 18° C. for 10 minutes. Then, the supernatant of the solution was sucked up and removed using an aspirator, and the blood cell medium was added and suspended to obtain a mononuclear cell suspension.

A solution in which 1.5 mL of PBS and 4.8 μL of silkworm-derived laminin (iMatrix-511 silk, nippi) were mixed was added to one well of a 6-well dish, and the dish was left in an incubator at 37° C. for 1 hour. Next, a solution in which PBS and laminin were mixed was removed from the well using an aspirator, and 1.5 mL of the mononuclear cell suspension was added to one well. The number of mononuclear cells in one well was 0.5×10⁵ to 5.0×10⁷. Then, the mononuclear cells were cultured in an incubator at 37° C. Some mononuclear cells were suspended.

Then, the medium in one well was replaced with a medium in which the 6 types of recombinant proteins were added to a serum-free medium for blood cells and an agent that alleviates a congenital antiviral response of cells was added. The amount of the medium replaced was 1.5 mL.

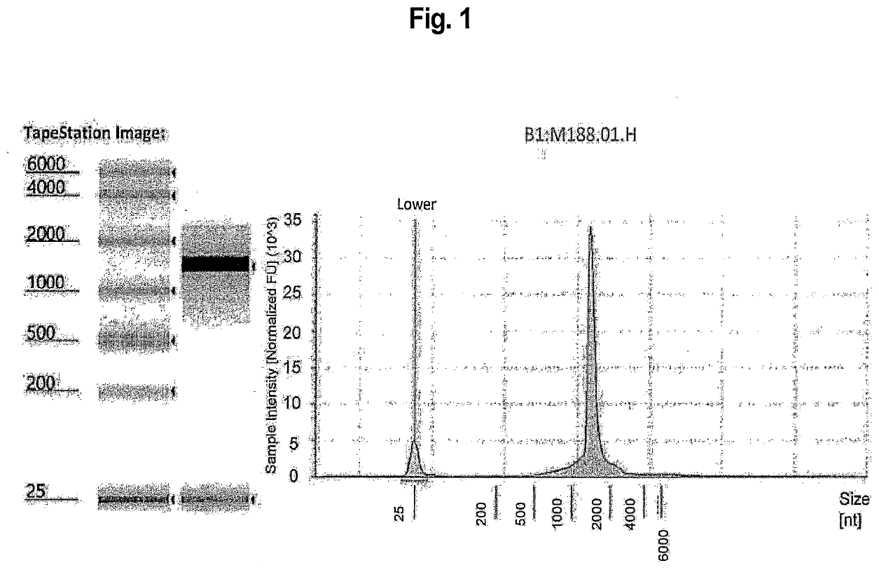

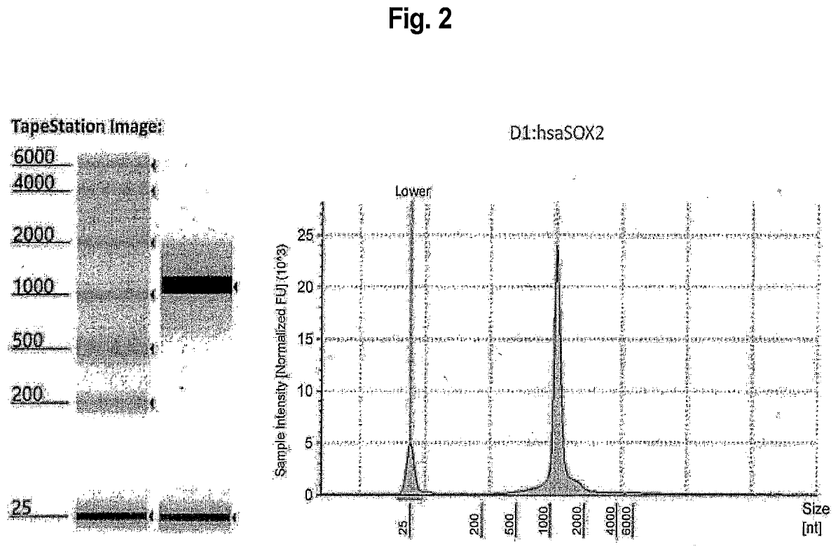

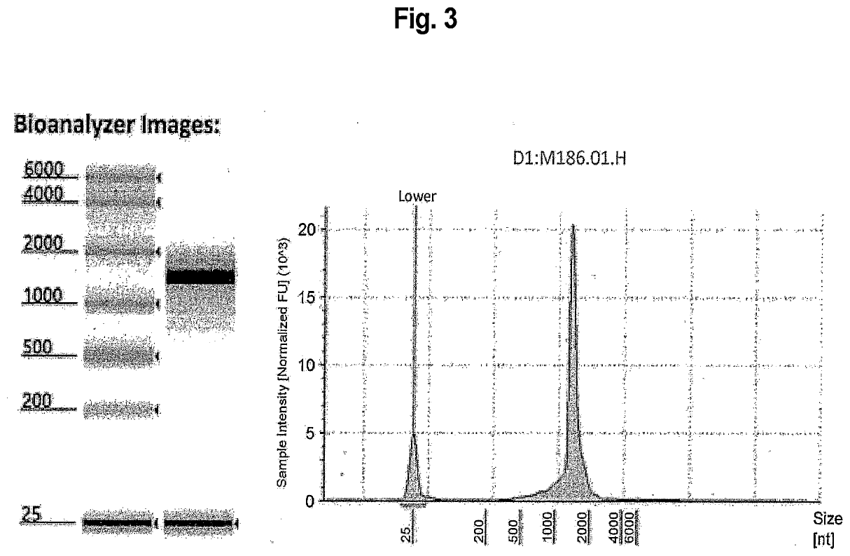

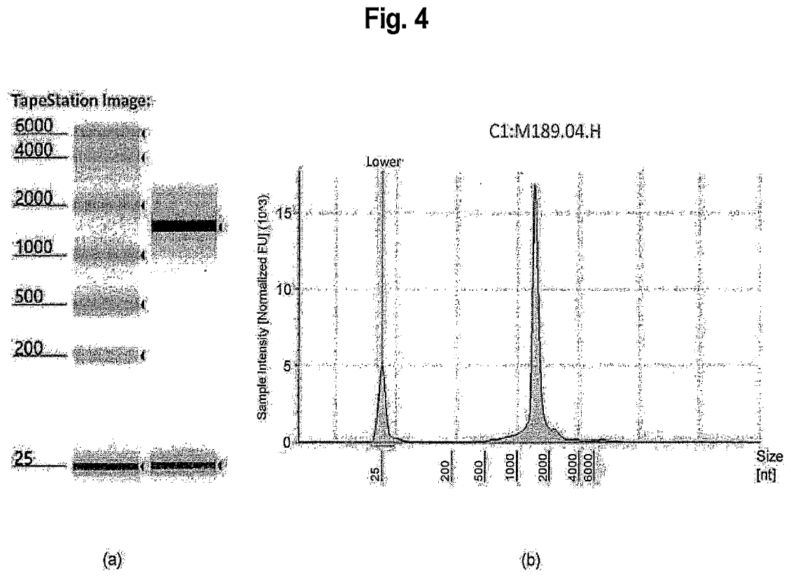

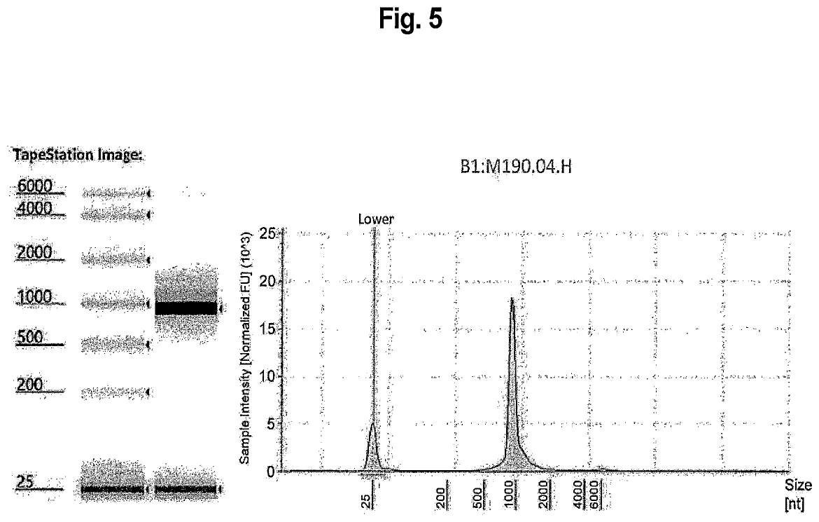

A tube A and a tube B were prepared, and 0.1 μL to 10² μL of a mixture containing M₃O RNA, SOX2 RNA, KLF4 RNA, C-MYC RNA, and LIN28 RNA (100 ng/μL) was added to 125 μL of PBS in the tube A. These RNAs were modified with pseudouridine (W). In addition, these RNAs were substantially concentrated and purified into single-stranded RNA through HPLC. It was confirmed that the ratio (A260/A280) of the absorbances of RNA at 260 nm and 280 nm was 1.71 to 2.1, and proteins were not substantially mixed. In addition, when dot blot analysis was performed using anti-double-stranded RNA antibody J2, 90% or more of double-stranded RNA was removed. FIG. 1 shows the results obtained by dot-blot analyzing M₃O RNA using anti-double-stranded RNA antibody J2, FIG. 2 shows the results obtained by dot-blot analyzing SOX2 RNA using anti-double-stranded RNA antibody J2, FIG. 3 shows the results obtained by dot-blot analyzing C-MYC RNA using anti-double-stranded RNA antibody J2, FIG. 4 shows the results obtained by dot-blot analyzing KLF4 RNA using anti-double-stranded RNA antibody J2, and FIG. 5 shows the results obtained by dot-blot analyzing LIN28 RNA using anti-double-stranded RNA antibody J2. 0.1 μL to 100 μL of a lipofection reagent was added to 125 μL of PBS in the tube B. Next, the solution in the tube A and the solution in the tube B were mixed, the mixed solution was left at room temperature for 10 minutes, and a total amount of the mixed solution was added to the blood cell medium in one well. Then, the dish was left in an incubator at 37° C. for 1 day, and the cells were transfected with RNA. Then, RNA transfection was repeated according to the same procedure for 5 to 7 days.

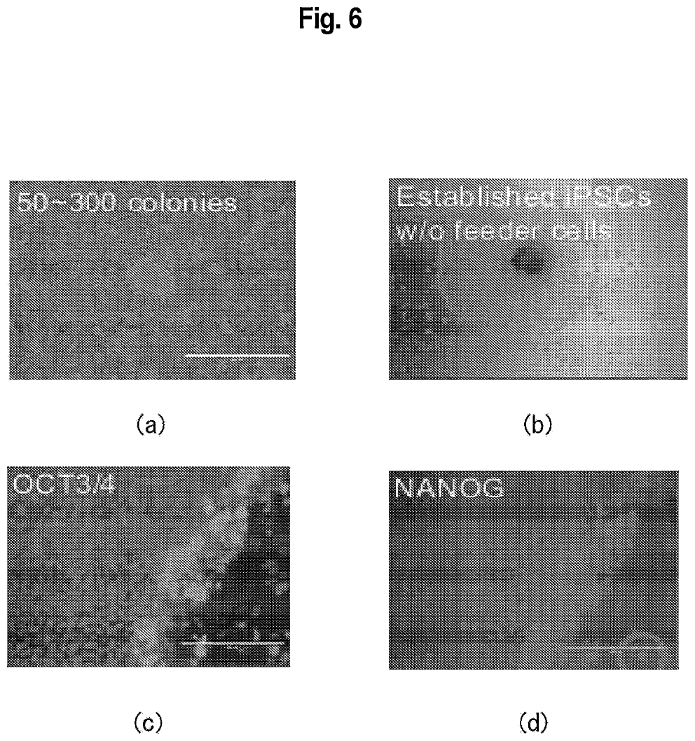

The blood cell medium in the well was replaced with 1.5 mL of a stem cell induction medium 8 to 10 days after the cells were seeded in the well. Then, RNA transfection was repeated until formation of iPS-cell-like colonies was observed as shown in FIG. 6(a) in the same procedures as above except that the medium was a stem cell induction medium. The initially suspended cells adhered to the wells over time.

When formation of iPS-cell-like colonies was observed 20 to 30 days after the cells were seeded in the wells, the iPS-cell-like colonies were collected from the wells, and in a stem cell maintenance medium (StemFit, Ajinomoto) in the laminin-coated dish, the cells were maintenance-cultured without using feeder cells. FIG. 6(b) shows an image of the maintenance-cultured cells. When the maintenance-cultured cells were immunostained with antibodies for OCT3/4, as shown in FIG. 6(c), the cells showed OCT3/4 positive. In addition, when the maintenance-cultured cells were immunostained with antibodies for NANOG, as shown in FIG. 6(d), the cells showed NANOG positive.

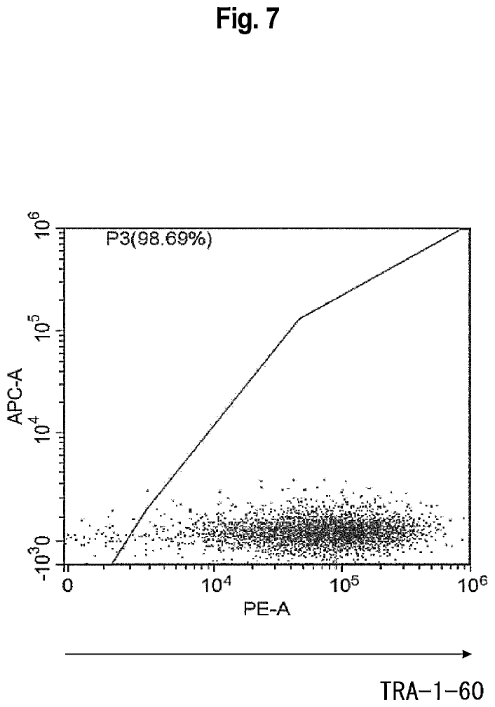

In addition, the maintenance-cultured cells were stained with antibodies labeled with an Allophycocyanin (APC) fluorescent dye, which are antibodies for TRA-1-60. Then, it was confirmed that the cells were TRA-1-60 positive using fluorescence-activated cell sorting (FACS (registered trademark), BD) as shown in FIG. 7.

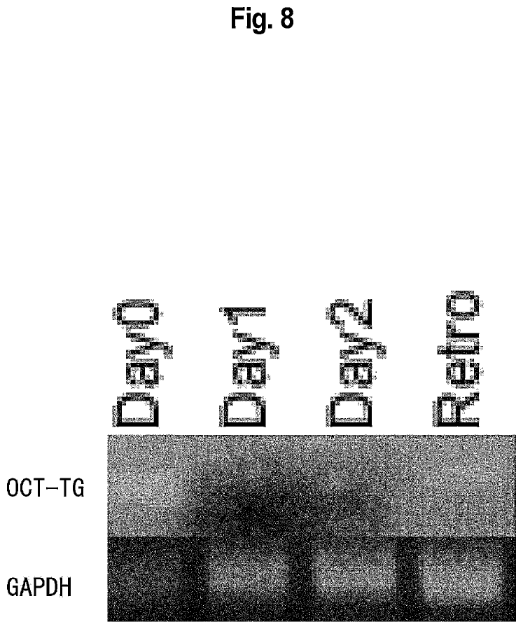

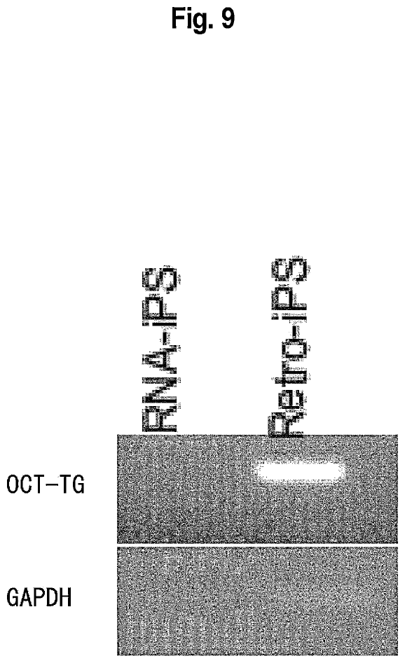

As shown in FIG. 8, when the cells transfected with RNA were analyzed through semi-quantitative PCR, it was confirmed that RNA transfected into the cell body no longer remained after 24 hours. In addition, as shown in FIG. 9, in iPS cells into which a reprogramming factor was introduced using retroviruses, OCT genes integrated into the genome were observed through semi-quantitative PCR (right lane), and in the RNA-lipofected cells according to this example, it was confirmed that OCT genes were not integrated into the genome (left lane).

### Comparative Example 1

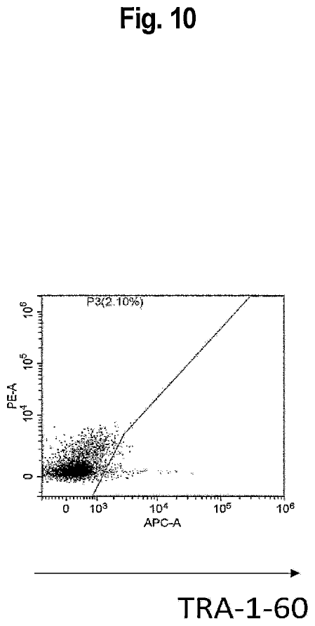

RNA was introduced into mononuclear cells in the same manner as in Example 1 except that RNA introduced into mononuclear cells was not substantially concentrated and purified into single-stranded RNA through HPLC. However, as shown in FIG. 10, the cells into which RNA was introduced did not become TRA-1-60 positive.

### Example 2

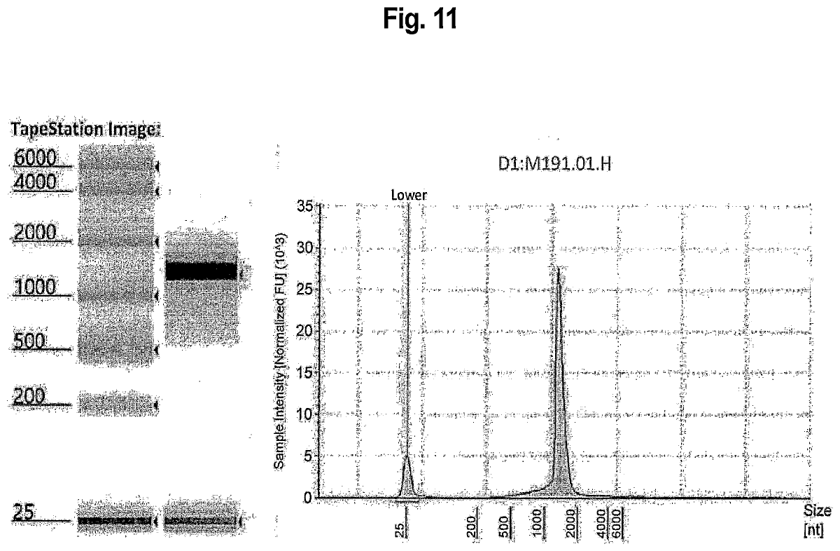

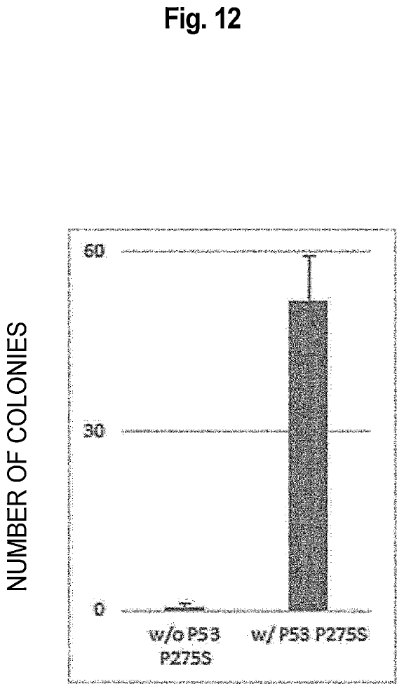

In addition to the RNA used in Example 1, RNA encoding p53P275S was transfected into mononuclear cells. RNA encoding p53P275S was also purified. FIG. 11 shows the results obtained by dot-blot analyzing p53P275S RNA using anti-double-stranded RNA antibody J2. As a result, as shown in FIG. 12, when RNA encoding p53P275S was transfected into mononuclear cells, more iPS-cell-like colonies were formed than when RNA encoding p53P275S was not transfected into mononuclear cells.

### Example 3

A DMEM containing 10% FBS was prepared as a medium for fibroblasts. Adult human-derived fibroblasts were suspended in a fibroblast medium to obtain a fibroblast suspension.

A solution in which 1.5 mL of PBS and 4.8 μL of silkworm-derived laminin (iMatrix-511 silk, nippi) were mixed was added to one well of a 6-well dish, and the dish was left in an incubator at 37° C. for 1 hour. Next, a solution in which PBS and laminin were mixed was removed from the well using an aspirator, and 1.5 mL of the fibroblast suspension was added to one well. The number of fibroblasts in one well was 0.5×10⁵ to 2.0×10⁵. Then, the fibroblasts were cultured in an incubator at 37° C. for 1 day.

Next, the medium was replaced with a stem cell induction medium. The amount of the medium replaced was 1.5 mL.

A tube A and a tube B were prepared, and 0.1 μL to 10² μL of a mixture containing RNA encoding p53P275S, M₃O RNA, SOX2 RNA, KLF4 RNA, C-MYC RNA, and LIN28 RNA (100 ng/μL) was added to 125 μL of PBS in the tube A. These RNAs were modified with pseudouridine (W). In addition, these RNAs were polyadenylated and capped. In addition, these RNAs were concentrated and purified through HPLC in the same manner as in Example 1. 0.1 μL to 100 μL of a lipofection reagent was added to 125 μL of PBS in the tube B. Next, the solution in the tube A and the solution in the tube B were mixed, the mixed solution was left at room temperature for 10 minutes, and a total amount of the mixed solution was added to the medium in one well. Then, the dish was left in an incubator at 37° C. for 1 day, and the cells were transfected with RNA. Then, RNA transfection was repeated for 5 to 9 days according to the same procedures. 11 days after the cells were seeded in the wells, the medium was replaced with a stem cell maintenance medium.

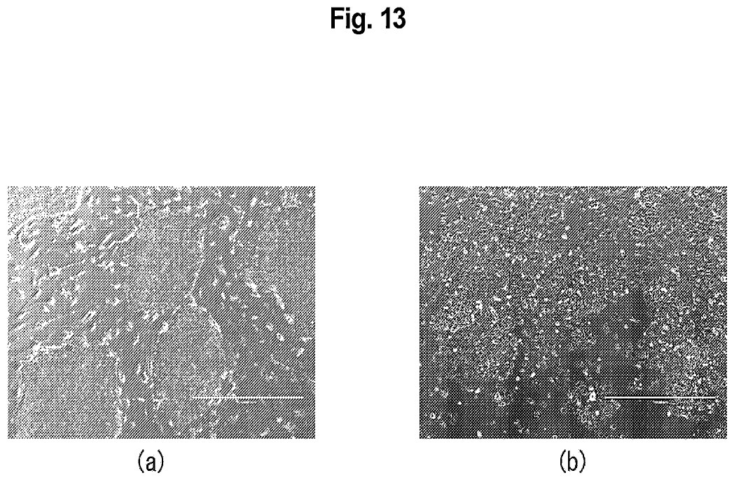

When formation of iPS-cell-like colonies was observed 5 to 9 days after the cells were seeded in the wells, as shown in FIG. 13(a), the iPS-cell-like colonies were collected from the wells, and in a stem cell maintenance medium in the laminin-coated dish, the cells were maintenance-cultured without using feeder cells. FIG. 13(b) shows an image of the maintenance-cultured cells.

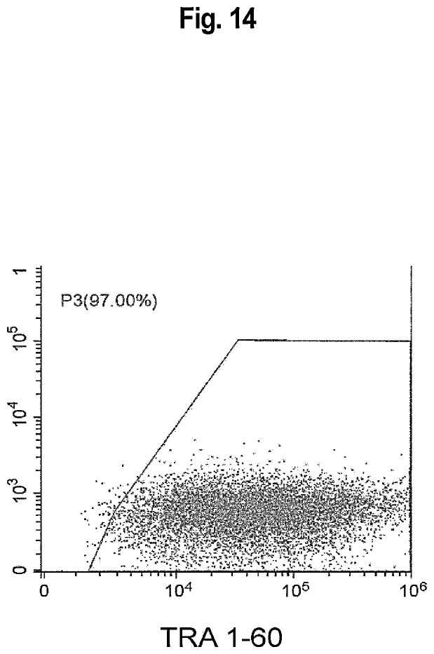

In addition, the maintenance-cultured cells were stained with antibodies labeled with an Allophycocyanin (APC) fluorescent dye, which are antibodies for TRA-1-60. Then, it was confirmed that the maintenance-cultured cells were TRA 60 positive using fluorescence-activated cell sorting (FACS (registered trademark), BD) as shown in FIG. 14.

### Comparative Example 2

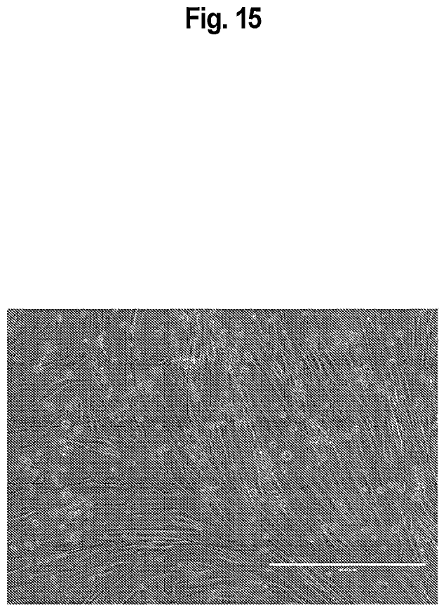

RNA was introduced into fibroblasts in the same manner as in Example 3 except that RNA introduced into mononuclear cells was not substantially concentrated and purified into single-stranded RNA through HPLC. However, as shown in FIG. 15, the cells into which RNA was introduced did not form iPS-cell-like colonies.

### Example 4

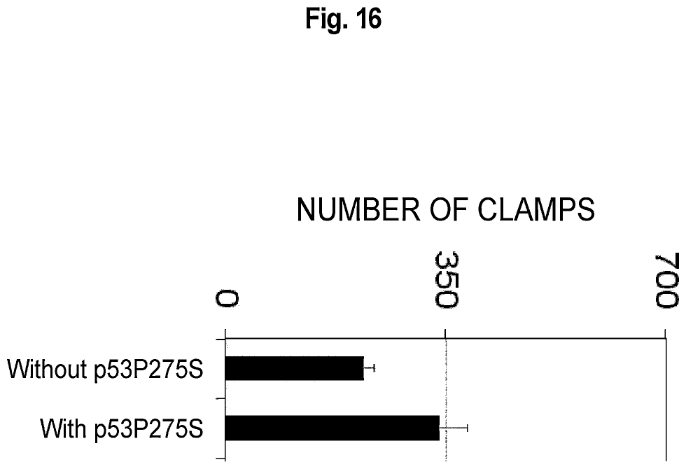

M₃O RNA, SOX2 RNA, KLF4 RNA, C-MYC RNA, and RNA encoding p53P275S were introduced into fibroblasts. Except for this, cells were induced in the same method as in Example 3, and as shown in FIG. 16, the efficiency of iPS-cell-like clamp formation was improved compared to the control in which RNA encoding p53P275S was not introduced.

### Example 5

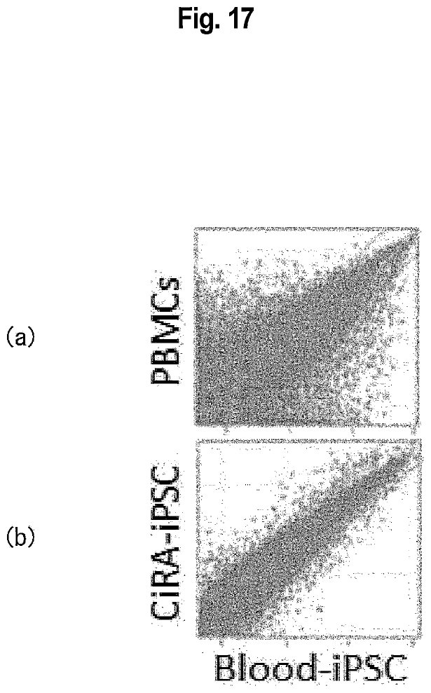

FIG. 17 shows the results of comprehensive comparison of gene expression between iPS-cell-like cells (Blood-iPSC) produced in Example 1 and iPS cells (CiRA-iPSC) produced at the Center for iPS Cell Research and Application, Kyoto University using a microarray. As shown in FIG. 17(a), the iPS-cell-like cells (Blood-iPSC) produced in Example 1 and peripheral blood mononuclear cells did not have similar global gene expression. On the other hand, as shown in FIG. 17(b), it was found that the global gene expression was very similar between the iPS-cell-like cells (Blood-iPSC) produced in Example 1 and iPS cells (CiRA-iPSC) produced at the Center for iPS Cell Research and Application, Kyoto University. Based on this result, it was confirmed that blood-derived iPS-cell-like cells derived from RNA had a gene expression profile very similar to the iPS cells derived so far.

### Example 6

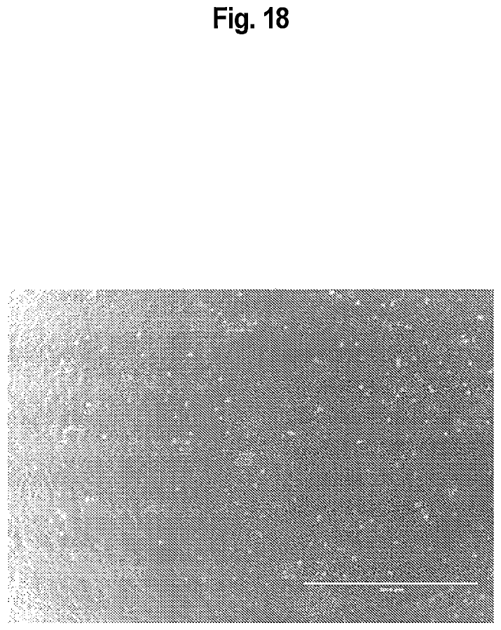

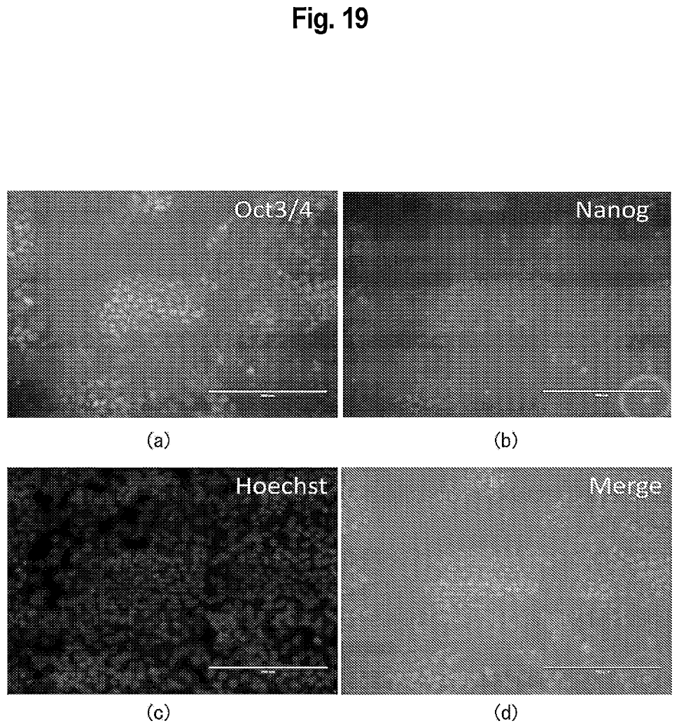

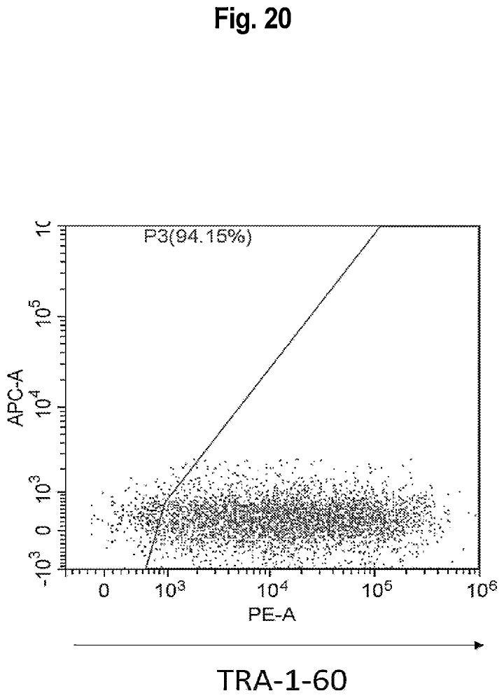

RNA was introduced into macaque-derived fibroblasts in the same method as in Example 3 except that macaque-derived fibroblasts were used. As a result, as shown in FIG. 18, formation of iPS-cell-like colonies was observed. When the maintenance-cultured cells were immunostained with antibodies for Oct3/4, as shown in FIG. 19(a), the cells showed Oct3/4 positive. In addition, when the maintenance-cultured cells were immunostained with antibodies for Nanog, as shown in FIG. 19(b), the cells showed Nanog positive. In addition, as shown in FIG. 20, it was confirmed that the induced cells were TRA-1-60 positive.

### Example 7

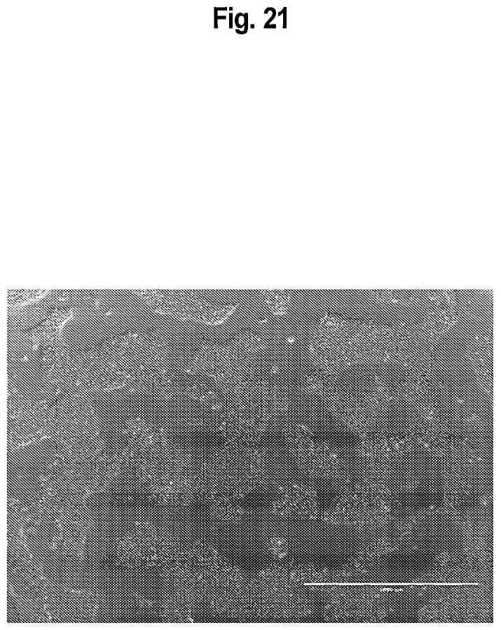

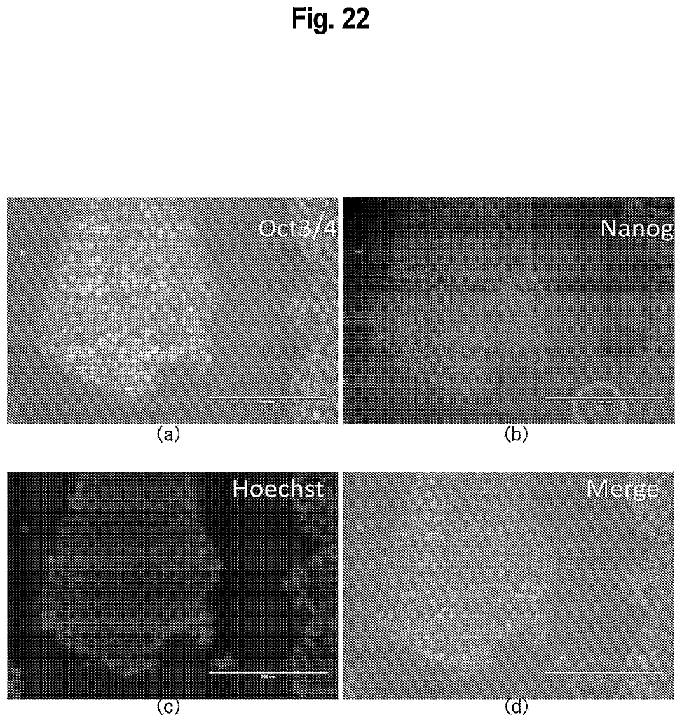

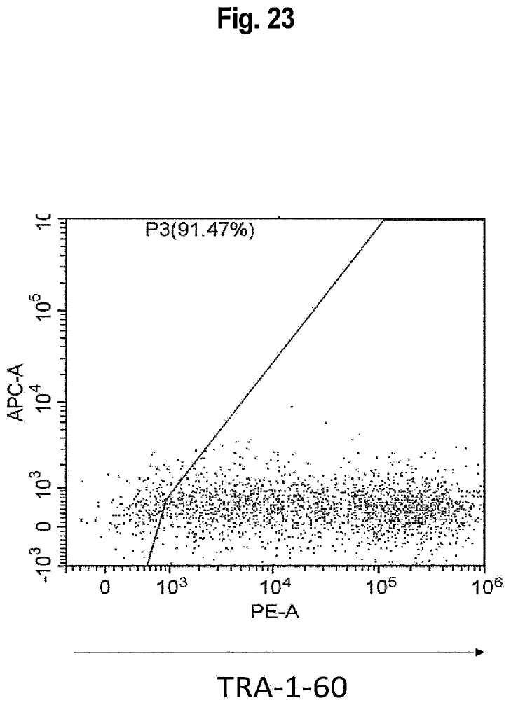

RNA was introduced into chimpanzee-derived fibroblasts in the same method as in Example 3 except that chimpanzee-derived fibroblasts were used. As a result, as shown in FIG. 21, formation of iPS-cell-like colonies was observed. When the maintenance-cultured cells were immunostained with antibodies for Oct3/4, as shown in FIG. 22(a), the cells showed Oct3/4 positive. In addition, when the maintenance-cultured cells were immunostained with antibodies for Nanog, as shown in FIG. 22(b), the cells showed Nanog positive. In addition, as shown in FIG. 23, it was confirmed that the cells were TRA-1-60 positive.

### Example 8

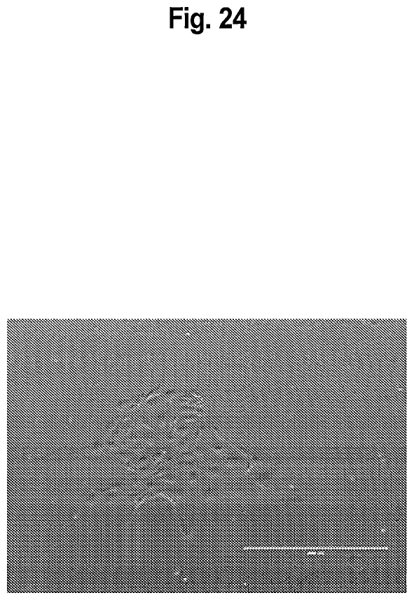

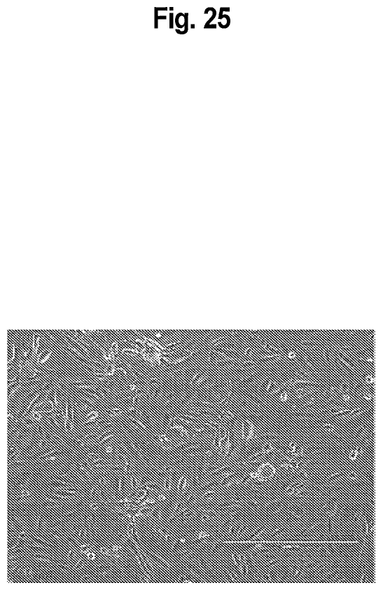

300 mL of urine was collected from a healthy subject, 6 urine samples were dispensed into a 50 mL Falcon tube, and the tube was centrifuged at 400G for 5 minutes. After centrifugation, the supernatant was removed from the tube, 30 mL of PBS was put into the tube, and the tube was centrifuged at 400G for 5 minutes. After centrifugation, the supernatant was removed from the tube, 30 mL of a primary medium was put into the tube, and the tube was centrifuged at 400G for 5 minutes. A primary medium was prepared by adding fetal bovine serum (Gibco, 10437028, final concentration 10%), SingleQuots Kit CC-4127 REGM (Lonza, 1/1000 amount), and Antibiotic-Antimycotic (Gibco, 15240062, 1/100 amount) to DMEM/Ham's F12 (Gibco, 11320-033). After centrifugation, the supernatant was removed from the tube, cells were suspended in 1 mL of the primary medium, the cells were seeded in one well of a gelatin-coated 24-well plate, and the cells were incubated in an incubator at 37° C. For 2 days after the cells were seeded, the primary medium was added to a 300 μL well, and from the 3rd day onward, the medium was replaced using a medium for epithelial cells. The medium for epithelial cells was prepared by adding SingleQuots Kit CC-4127 REGM (Lonza) to a renal epithelial cell basal medium (Lonza). FIG. 24 shows a microscope image of the cells after expansion-cultured for 6 days. The cells were subjected to the first passage on the 7th day after seeding, the cells were additionally expansion-cultured, and the cells were subjected to the second passage on the 7th day after the first passage. FIG. 25 shows a microscope image of the cells on the 6th day after the second passage.

### Example 9

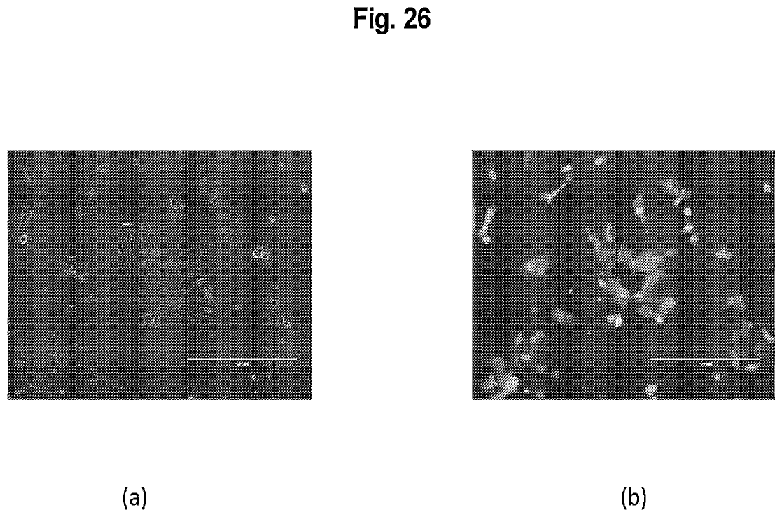

A dish coated with laminin 511 was used as a dish for inducing pluripotent stem cells. 1×10⁴ to 1×10⁵ urine-derived cells prepared in Example 8 were seeded in the dish for inducing pluripotent stem cells and incubated at 37° C. For the medium, a medium for epithelial cells was used. The next day, a mixture containing a transfection reagent and RNA that encodes a green fluorescent protein (GFP) was added to a medium, the medium was replaced with the above medium, and incubated at 37° C. FIG. 26 shows microscope images of the cells on the next day. Expression of GFP was observed, which indicates that transfection into urine-derived cells was performed.

### Example 10

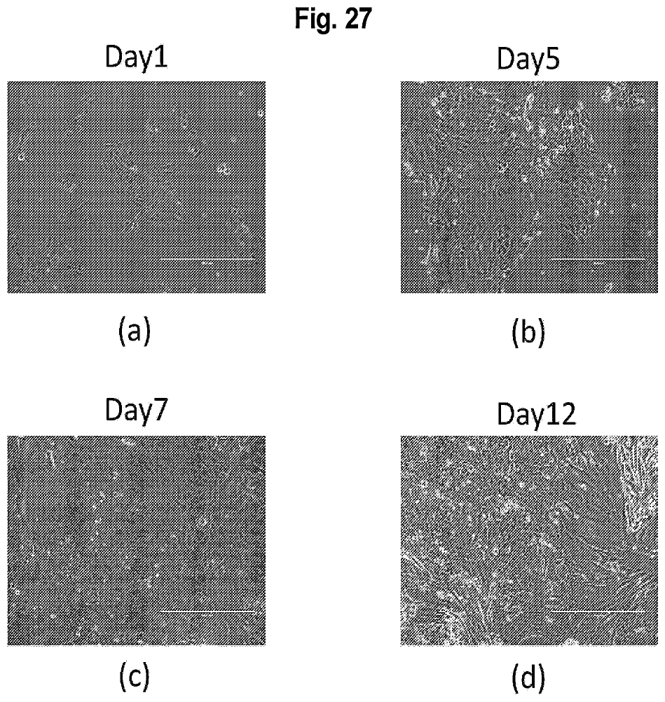

A dish coated with laminin 511 was used as a dish for inducing pluripotent stem cells. 1×10⁴ to 1×10⁵ urine-derived cells prepared in Example 8 were seeded in the dish for inducing pluripotent stem cells and incubated at 37° C. For the medium, a medium for epithelial cells was used. The next day, a tube A and a tube B were prepared, and 0.1 μL to 10² μL of a mixture containing M₃O RNA, SOX2 RNA, KLF4 RNA, C-MYC RNA, and LIN28 RNA (100 ng/μL) was added to 125 μL of PBS in the tube A. These RNAs were modified with pseudouridine (W). In addition, these RNAs were substantially concentrated and purified into single-stranded RNA through HPLC. It was confirmed that the ratio (A260/A280) of the absorbances of RNA at 260 nm and 280 nm was 1.71 to 2.1, and proteins were not substantially mixed. In addition, when dot blot analysis was performed using anti-double-stranded RNA antibody J2, 90% or more of double-stranded RNA was removed. 0.1 μL to 100 μL of a lipofection reagent was added to 125 μL of PBS in the tube B. Next, the solution in the tube A and the solution in the tube B were mixed, the mixed solution was left at room temperature for 10 minutes, a total amount of the mixed solution was added to a transfection medium without using B18R or the like, the medium was replaced using the transfection medium, and incubated at 37° C. Transfection was performed once daily for 10 days. Observation was performed on days 1, 5, 7, and 14 after cell seeding. As shown in FIG. 27, it was observed that cell morphology changed like ES cells as the day progressed.

### Example 11

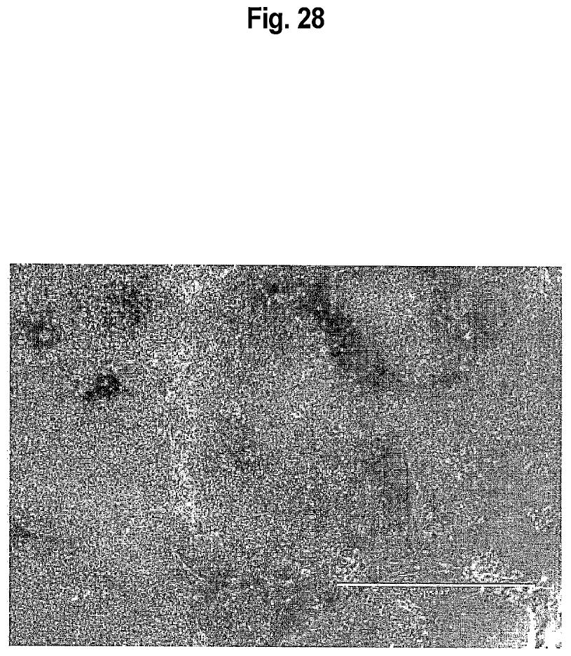

As in Example 10, urine-derived cells were transfected for 10 days. The cells were cultured in a medium for stem cells (StemFit, Ajinomoto) from the 11th day after the cells were seeded, all cells were separated from the dish on the 14th day, and some of the recovered and mixed cells were seeded and passaged in the medium. During passage, without picking up colonies, all cells on the dish were recovered, and 1×10² to 1×10⁵ cells were seeded on the dish. FIG. 28 shows a microscope image of the cells 6 days after passage. ES-cell-like cells were confirmed.

### Example 12

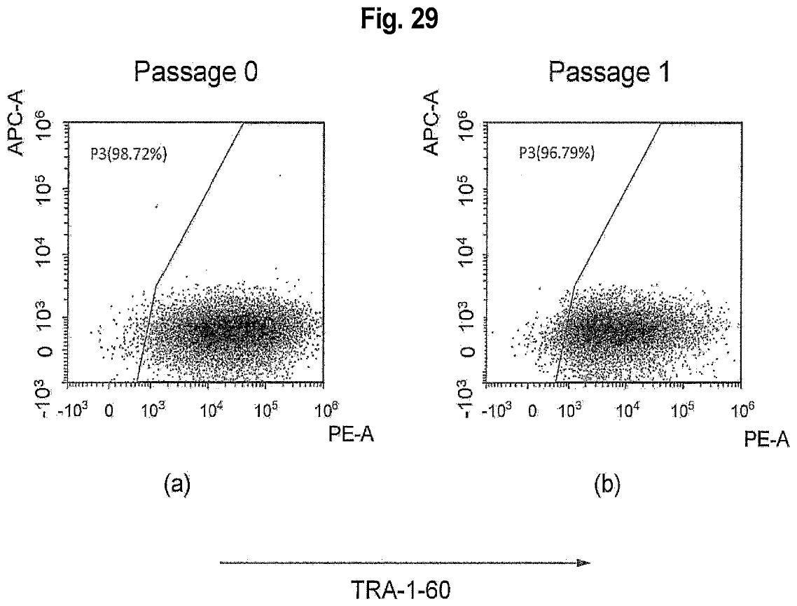

As in Example 10, urine-derived cells were transfected for 10 days. When the cells were cultured in a medium for stem cells (StemFit, Ajinomoto) from the 11th day after the cells were seeded, the cells were separated from the dish on the 14th day, and some of the cells were analyzed using a flow cytometry, as shown in FIG. 29(a), TRA-1-60 positive was confirmed. In addition, when the cells separated from the dish on the 14th day were passaged, and analyzed using a flow cytometry 7 days later, as shown in FIG. 29(b), TRA-1-60 positive was confirmed.

### Example 13

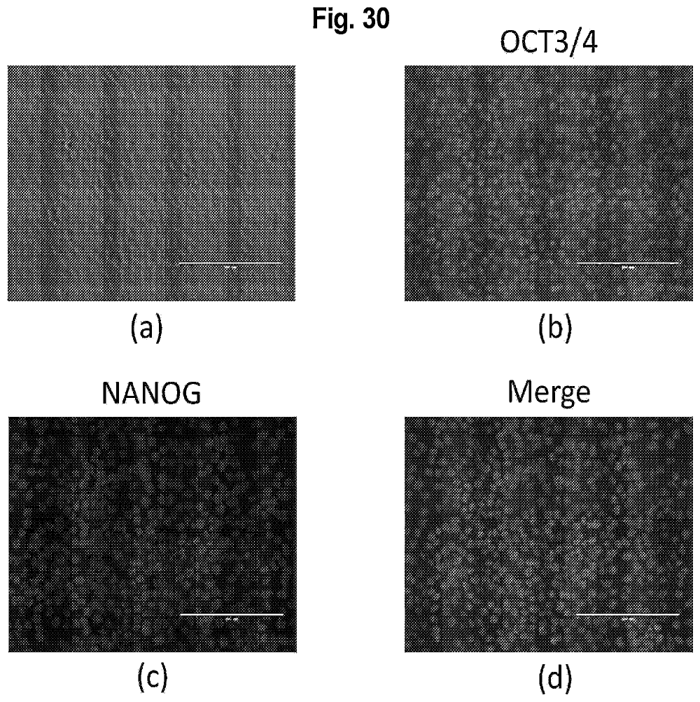

As in Example 10, urine-derived cells were transfected for 10 days. The cells were cultured in a medium for stem cells (StemFit, Ajinomoto) from the 11th day after the cells were seeded, all cells were separated from the dish on the 14th day, and some of the separated and mixed cells were seeded and passaged. StemFit (registered trademark) was used as the medium after passage. 7 days after passage, cells were immobilized, and the cells were stained using anti-OCT3/4 antibodies and anti-NANOG antibodies. In addition, nuclei chemical staining using Hoechst (registered trademark) was performed. As a result, as shown in FIG. 30, expression of OCT3/4 and NANOG, which are specific markers for pluripotent stem cells, in cell nuclei was confirmed. Therefore, it was shown that the pluripotent stem cells could be induced from urine-derived cells using RNA. Here, FIG. 30(d) is an image obtained by synthesizing an image of cells stained using anti-OCT3/4 antibodies, an image of cells stained using anti-NANOG antibodies, and an image of cells stained using Hoechst (registered trademark).

### Example 17

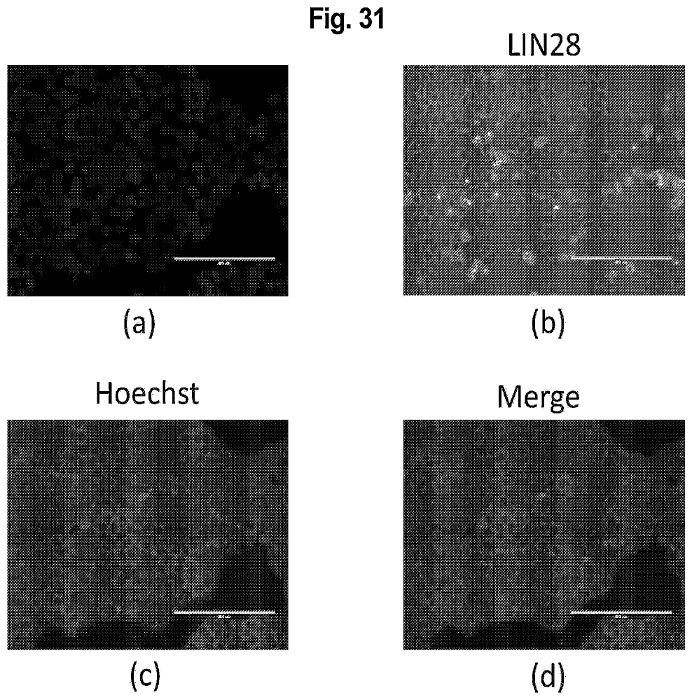

As in Example 10, urine-derived cells were transfected for 10 days. The cells were cultured in a medium for stem cells (StemFit, Ajinomoto) from the 11th day after the cells were seeded, all cells were separated from the dish using a TrypLE select on the 14th day, and some of the separated and mixed cells were seeded and passaged in a medium. StemFit (registered trademark) was used as the medium after passage. 7 days after passage, cells were immobilized, and the cells were stained using anti-LIN28 antibodies. In addition, nuclei chemical staining using Hoechst (registered trademark) was performed. As a result, as shown in FIG. 31, expression of LIN28, which is a specific marker for pluripotent stem cells, in cell nuclei was confirmed. Therefore, it was shown that the pluripotent stem cells could be induced from urine-derived cells using RNA. Here, FIG. 31(d) is an image obtained by synthesizing an image of cells stained using anti-LIN28 antibodies and an image of cells stained using Hoechst (registered trademark).

1. A method for manufacturing induced pluripotent stem cells, comprising:
preparing cells; and
introducing RNA into the cells,
wherein the RNA includes RNA encoding a reprogramming factor, and
wherein, in the RNA, double-stranded RNA is substantially removed.

2. The method according to claim 1,
wherein the RNA is purified through HPLC.

3. The method according to claim 1,
wherein the RNA further includes RNA encoding a dominant negative mutant of p53.

4. The method according to claim 1,
wherein the RNA encoding the reprogramming factor includes at least one selected from the group consisting of OCT3/4 RNA, SOX2 RNA, KLF4 RNA, and C-MYC RNA.

5. The method according to claim 1,
wherein the RNA encoding the reprogramming factor includes OCT3/4 RNA and RNA of a transactivation domain (TAD) of MYOD connected to the OCT3/4 RNA.

6. The method according to claim 1,
wherein an introduction of the RNA into the cells is performed by a lipofection method.

7. The method according to claim 1,
wherein, in the introduction, the cells are adhered to a substrate.

8. The method according to claim 7,
wherein the substrate is coated with a matrix in which induced pluripotent stem cells are able to be cultured.

9. The method according to claim 1,
wherein the cells are fibroblasts.

10. The method according to claim 1,
wherein the cells are blood cells.

11. The method according to claim 10,
wherein the blood cells are not expansion-cultured endothelial progenitor cell.

12. The method according to claim 10,
wherein, before the RNA is introduced, the blood cells are expansion-cultured in a blood cell medium for 10 days or less.

13. The method according to claim 10,
wherein the blood cells are at least one selected from the group consisting of mononuclear cells, T cells, B cells, monocytes, macrophages, blood stem cells, dendritic cells, and granulocytes.

14. The method according to claim 10,
wherein the blood cells are derived from peripheral blood or cord blood.

15. The method according to claim 10,
wherein the blood cells are derived from adult blood.

16. The method according to claim 10, further comprising culturing the blood cells into which the RNA is introduced in a blood cell medium and then culturing in a stem cell induction medium.

17. The method according to claim 1,
wherein the cells are cells contained in urine.

18. The method according to claim 1,
wherein the cells are bladder epithelial cells.

19. The method according to claim 17, further comprising collecting cells into which the RNA is introduced from urine.

20. The method according to claim 1,
wherein the cells are derived from humans.

21. The method according to claim 1,
wherein the cells are derived from non-human primate animals.
